(19) United States
(12) Patent Application Publication
(10) Pub. No.: US 2024/0366201 A1
(43) Pub. Date: Nov. 7, 2024

(54) MEDICAL DEVICES AND RELATED METHODS FOR CONTROLLING AND/OR LOCKING A NEEDLE

(71) Applicant: Boston Scientific Scimed, Inc., Maple Grove, MN (US)

(72) Inventors: Gonzalo Jose Saenz VILLALOBOS, Alajuela (CR); Fernando Valerio GUERRERO, Alajuela (CR); Esteban de la Trinidad Jiménez SÁNCHEZ, San Jose (CR)

(73) Assignee: Boston Scientific Scimed, Inc., Maple Grove, MN (US)

(21) Appl. No.: 18/653,276

(22) Filed: May 2, 2024

Related U.S. Application Data
(60) Provisional application No. 63/499,807, filed on May 3, 2023.

(51) Int. Cl. A61B 17/00

(52) U.S. Cl. CPC A61B 17/00234; A61B 2017/00296; A61B 2017/00367; A61B 2017/00477; A61B 2217/007

(57) ABSTRACT

A medical device includes a handle with a movable portion and a stationary portion. The medical device also includes a shaft extending from a distal portion of the handle, and a distal portion of the shaft includes a needle. Movement of the movable portion of the handle relative to the stationary portion of the handle controls an extension or retraction of the needle. The movable portion includes a tab, and a position of the tab relative to the stationary portion releasably secures a position of the movable portion relative to the stationary portion and also secures a position of the needle relative a distal end of the shaft.

### CROSS-REFERENCE TO RELATED APPLICATIONS

This application claims the benefit of priority of U.S. Provisional Patent Application No. 63/499,807, filed on May 3, 2023, the entirety of which is incorporated herein by reference.

### TECHNICAL FIELD

Various aspects of this disclosure generally relate to medical devices and related methods for controlling and/or locking a needle. Embodiments of the disclosure relate to medical devices and related methods of treating tissue by delivering a needle to or into tissue.

### BACKGROUND

Medical devices, such as endoscopes or other suitable insertion devices, are employed for a variety of types of diagnostic and surgical procedures, such as endoscopy, laparoscopy, arthroscopy, gynoscopy, thoracoscopy, cystoscopy, etc. Many of these procedures involve injecting or otherwise delivering fluid to or into tissue of an organ or a gland to treat lesions (e.g., tumors), infections, and the like. Examples of such procedures include Endoscopic Mucosal Resection (EMR), Endoscopic Sub-mucosal Resection (ESR), Endoscopic Sub-mucosal Dissection (ESD), polypectomy, mucosectomy, Peroral Endoscopic Myotomy (POEM), etc. In particular, such procedures may be carried out by inserting an insertion device into a subject's body through a surgical incision, or via a natural anatomical orifice (e.g., mouth, vagina, or rectum), and performing the procedure or operation at a treatment site with an auxiliary device inserted through the insertion device. Alternatively, the auxiliary device may be delivered to the treatment site without the insertion device.

In some procedures, a distal portion of a medical device (e.g., an auxiliary device) is positioned into tissue. In some aspects, the medical device may be a needle, for example, with a sharp distal end. A proximal end of the medical device may be coupled to one or more syringe or other fluid sources, such that fluid is delivered through the needle to, for example, inject the fluid into or otherwise around tissue at the treatment site. The needle may be movable, for example, extendable distally and retractable proximally. In existing devices, a user may be required to maintain the proximal portion (e.g., a handle) in order to maintain the needle in the extended configuration. Alternatively, some medical devices include locking devices to maintain the needle in the extended position, but such locking devices may be difficult to understand, difficult to lock in the extended configuration, or otherwise difficult to operate. These concerns may increase the duration, costs, and risks of the medical procedure.

The devices and methods of this disclosure may rectify some of the deficiencies described above or address other aspects of the art.

### SUMMARY

Examples of this disclosure relate to, among other things, medical devices and methods for performing one or more medical procedures. For example, this disclosure relates to medical devices and methods for performing one of more procedures at a treatment site, for example, between layers of tissue. Additionally, in some examples, this disclosure relates to medical devices and methods for delivering one or more fluids to the treatment site. Furthermore, in some examples, this disclosure relates to medical devices and methods for extending, retracting, and/or securing one or more portions of a handle, and correspondingly, extending, retracting, and/or securing a configuration of a needle. Each of the examples disclosed herein may include one or more of the features described in connection with any of the other disclosed examples.

In one example, a medical device may include a handle with a movable portion and a stationary portion. The medical device also may include a shaft extending from a distal portion of the handle, and a distal portion of the shaft includes a needle. Movement of the movable portion of the handle relative to the stationary portion of the handle may control an extension or retraction of the needle. The movable portion may include a tab, and a position of the tab relative to the stationary portion may releasably secure a position of the movable portion relative to the stationary portion and also may secure a position of the needle relative a distal end of the shaft.

The medical device may include one or more of the following features. The stationary portion may include a track to movably receive a portion of the tab. The track may include one or more locking portions. The medical device may further include a trigger, and the trigger may be pivotable relative to the stationary portion of the handle. The trigger may be configured to interact with the tab of the movable portion to limit proximal movement of the movable portion. The medical device may further include one or more springs, and the one or more springs may bias the distal movement of the movable portion relative to the stationary portion of the handle. The movable portion may further include a first grip portion configured to accept an applied force and the applied force may cause the movable portion to move relative to stationary portion. The movable portion may be disposed on a first face. The stationary portion may further include a second grip portion disposed on a second face, the second face opposite the first face. The tab may be circular and extend radially from an extension portion. The one or more locking portions may include a circular opening configured to engage the tab. Force applied to the tab may cause the tab to release from the circular opening. The tab may include a cantilevered projection disposed within an opening on the stationary portion. A maximal extension of the needle may correspond to a snap-fit between the tab and the opening. The movable portion may be configured to mate the stationary portion. The one or more springs may be biased by one or more of the movable portion, the stationary portion, or a trigger. The trigger may be pivotable relative to the stationary portion of the handle.

In another example, a medical device may include a handle with a movable portion and a stationary portion. The medical device may further include a shaft extending from a distal portion of the handle. A distal portion of the shaft may include a needle. Movement of the movable portion of the handle relative to the stationary portion of the handle may control a position of the needle. The movable portion may include a tab. A position of the tab relative to the stationary portion may releasably lock a position of the movable portion relative to the stationary portion.

The medical device may include one or more of the following features. The tab may further lock a position of the needle relative a distal end of the shaft. The medical device may further include a trigger. The trigger may be pivotable relative to the stationary portion of the handle. The handle may include a depression configured to accept the trigger. The trigger may include a prong configured to engage the tab.

In yet another example, a medical device may include a handle with a movable portion including a knob and a stationary portion. The medical device may further include a shaft extending from a distal portion of the handle. A distal portion of the shaft may include a needle. Movement of the movable portion of the handle relative to the stationary portion of the handle may control an extension or retraction of the needle. The movable portion may include a tab. A position of the tab relative to the stationary portion may releasably secure a position of the movable portion relative to the stationary portion and also may secure a position of the needle relative a distal end of the shaft. Rotation of the knob may rotate the movable portion relative to the stationary portion.

The medical device may include one or more of the following features. The stationary portion may include a track to movably receive a portion of the tab. The track may be angled relative to a longitudinal axis of the stationary portion.

It may be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIGS. 1A and 1B illustrate perspective views of a medical device in an unlocked configuration and a locked configuration, respectively, according to aspects of this disclosure.

FIGS. 2A and 2B illustrate perspective views of another medical device in an unlocked configuration and a locked configuration, respectively, according to aspects of this disclosure.

FIGS. 3A and 3B illustrate perspective views of yet another medical device in an unlocked configuration, with FIG. 3B including some features as partially transparent, and FIG. 3C illustrates a perspective view of the medical device in a locked configuration, according to aspects of this disclosure.

FIGS. 4A and 4B illustrate perspective views of a further medical device in an unlocked configuration, with FIG. 4B including some features as partially transparent, and FIG. 4C illustrates a perspective view of the medical device in a locked configuration, according to aspects of this disclosure.

FIGS. 4D-4G illustrate partially transparent views of a portion of the medical device of FIGS. 4A-4C in various configurations, according to aspects of this disclosure.

FIGS. 5A-5C illustrate various views of another medical device in various configurations, according to aspects of this disclosure.

FIGS. 6A-6D illustrate various views of an additional medical device in various configurations, according to aspects of this disclosure.

### DETAILED DESCRIPTION

Examples of the disclosure include devices and methods for one or more of: facilitating and improving the efficacy, efficiency, and safety of treating and/or manipulating tissue when, for example, delivering a needle to tissue; delivering fluid through the needle into, under, and/or around tissue during a medical procedure through the needle; or otherwise treating tissue. Aspects of the disclosure may provide the user with the ability to control the position of a needle, for example, via a handle that remains proximal to the patient. Aspects of this disclosure may provide the user with the ability to lock or otherwise releasably secure the position of the needle via the handle, for example, with the needle in an extended or deployed configuration or with the needle in a withdrawn or retracted configuration. Aspects of this disclosure may allow for the user to releasably lock or secure the position of the needle (e.g., via operation of the handle) with one hand. Aspects of the disclosure may provide the user with the ability to easily determine a configuration (e.g., deployed or retracted) of the needle, for example, by one or more indications on the proximal handle. The operation and use of aspects of this disclosure may be intuitive and/or easy to use.

Moreover, aspects of this disclosure may help to shield the needle and/or otherwise help to provide safe operation of the medical device. For example, aspects of this disclosure may help to shield the needle during delivery or positioning at a treatment site. In another example, aspects of this disclosure may help to provide a safety mechanism, for example, automatically retracting the needle from an extended position when the user repositions one or more portions of the handle in order to unlock the needle from the extended configuration. Furthermore, some aspects of this disclosure may help to provide for ergonomic and/or comfortable actuation and/or handling for the one or more users. Some aspects of the disclosure may be used in performing an endoscopic, laparoscopic, arthroscopic, gynoscopic, thoracoscopic, cystoscopic, or other type of procedure.

Embodiments of the disclosure may relate to devices and methods for performing various medical procedures and/or treating portions of the large intestine (colon), small intestine, cecum, esophagus, stomach, or any other portion of the gastrointestinal tract, lungs, and/or any other suitable patient anatomy. Various embodiments described herein include single-use or disposable medical devices. Some aspects of the disclosure may be used in performing an endoscopic, arthroscopic, bronchoscopic, ureteroscopic, colonoscopic, or other type of procedure. For example, the disclosed aspects may be used with duodenoscopes, bronchoscopes, ureteroscopes, colonoscopes, catheters, diagnostic or therapeutic tools or devices, or other types of medical devices. One or more of the elements discussed herein could be metallic, plastic, or include a shape memory metal (such as, e.g., nitinol), a shape memory polymer, a polymer, or any combination of biocompatible materials.

The terms “proximal” and “distal” are used herein to refer to the relative positions of the components of an exemplary medical device. When used herein, “proximal” refers to a position relatively closer to the exterior of the body of a subject or closer to a user, such as a medical professional, holding or otherwise using the medical device. In contrast, “distal” refers to a position relatively further away from the medical professional or other user holding or otherwise using the medical device, or closer to the interior of the subject's body. Proximal and distal directions are labeled with arrows marked “P” and “D”, respectively, throughout various figures. As used herein, the terms “comprises,” “comprising,” “having,” “including,” or other variations thereof, are intended to cover a non-exclusive inclusion, such that a device or method that comprises a list of elements does not include only those elements, but may include other elements not expressly listed or inherent thereto. Unless stated otherwise, the term “exemplary” is used in the sense of “example” rather than “ideal.” As used herein, the terms “about,” “substantially,” and “approximately,” indicate a range of values within +/−10% of a stated value.

Reference will now be made in detail to examples of the disclosure described above and illustrated in the accompanying drawings. Wherever possible, the same reference numbers will be used throughout the drawings to refer to the same or like parts. It is noted that one or more aspects of the medical devices or methods discussed herein may be combined and/or used with one or more other aspects of the medical devices or methods discussed herein.

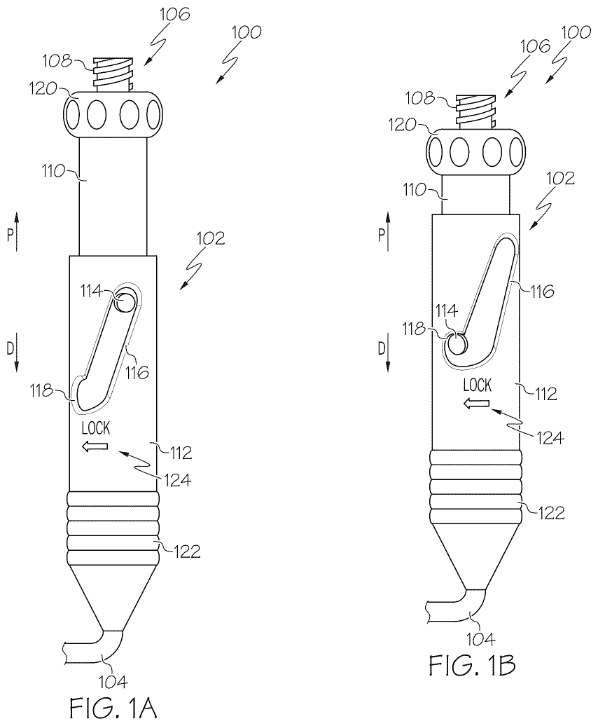

FIGS. 1A and 1B illustrate perspective views of an exemplary medical device 100 that includes a handle 102 and a shaft 104. Although not shown, shaft 104 includes a distal end, and medical device 100 further includes a movable needle (also not shown), which may be movable relative to the distal end of shaft 104. Although a needle is disclosed herein, it will be appreciated that the handles described herein may be used to transition other types of elements of medical devices (e.g., end effectors). As discussed in detail below, one or more portions of handle 102 are movable to control the movement and/or position of the needle, for example, movable between a retracted position (FIG. 1A) and an extended position (FIG. 1B). Although not shown, one or more portions of handle 102, shaft 104, and/or the needle may include one or more lumens or channels, for example, to deliver one or more fluids from one or more fluid sources (e.g., coupled to a proximal end of handle 102), through medical device 100, and out of the needle. For example, a proximal portion of handle 102 may include a coupling portion 106. Coupling portion 106 may be a port, a luer, etc., for example, including one or more threads 108, to couple a syringe or other fluid source to medical device 100.

As shown in FIGS. 1A and 1B, handle 102 includes an internal or movable portion 110 and an external or stationary portion 112. Movement of movable portion 110 relative to stationary portion 112 may control the position of the needle relative to the distal end of shaft 104. For example, movable portion 110 may include a projection or tab 114, and stationary portion 112 may include a channel or track 116, for example, through which tab 114 may move as movable portion 110 moves relative to stationary portion 112. Track 116 maybe formed by an opening (e.g., carved or otherwise extending radially through) in one or more portions of stationary portion 112 of handle 102. One or more portions of track 116 may extend at an angle relative to a longitudinal axis of stationary portion 112, for example, spanning a portion of a circumference of stationary portion 112. As shown in FIGS. 1A and 1B, track 116 may extend at an angle such that track 116 extends more in a longitudinal direction (approximately parallel to a longitudinal axis of stationary portion 112) than in a circumferential direction around a portion of a circumference of stationary portion 112. However, such an arrangement is merely exemplary. The interaction of tab 114 within track 116 may limit the longitudinal and/or rotational movement of movable portion 110 relative to stationary portion 112 of handle.

Moreover, track 116 (e.g., a distal portion of track 116) may include one or more locking portions 118, for example, formed by a widened portion (in a circumferential) or protrusion in track 116. Locking portion(s) 118 may be include a partially circular portion, for example, with a semi- or partially-circular portion or otherwise curved portion, with the opening of this portion facing at least partially toward a distal end of handle 102. In these aspects, locking portion(s) 118 may include a shape that at least partially corresponds to a shape of tab 114. Track 116 may have a shape similar to a golf club or a checkmark, with a distal portion of track 116 protruding circumferentially from a more proximal portion of track 116. As such, positioning tab 114 in locking portion(s) 118 may help to releasably secure movable portion 110 relative to stationary portion 112, for example, to releasably secure the needle in an extended configuration.

Furthermore, although not shown, movement of movable portion 110 relative to stationary portion 112 may engage one or more springs, for example, biasing the distal movement of movable portion 110, for example, biasing movable portion 110 proximally. For example, as movable portion 110 moves distally relative to stationary portion 112, the one or more springs may be compressed. With tab 114 in locking portion(s) 118, the position of movable portion 110 may be at least partially secured relative to stationary portion 112. Then, when movable portion 110 is manipulated (e.g., rotated or extended slightly distally) such that tab 114 is moved out of locking portion(s) 118, the one or more springs may bias movable portion 110 to move from the extended configuration (FIG. 1B) to the retracted configuration (FIG. 1A) (e.g., may bias movable portion 110 to move in a proximal direction).

In some aspects, movable portion 110 may include one or more knob portions 120, for example, on a proximal portion of movable portion 110 (e.g., adjacent to coupling portion 106). Knob portion(s) 120 may help the user grip and/or otherwise manipulate (e.g., extend/retract, rotate, etc.) movable portion 110. In some examples, a user may use his or her thumb to press on knob portion 120 in order to extend/retract movable portion 110. Furthermore, in some aspects, stationary portion 112 may include one or more grip portions 122, for example, on a distal portion of stationary portion 112. Grip portion(s) 122 may help the user grip and/or hold stationary portion 112.

Additionally, in some aspects, stationary portion 112 may include one or more indications 124, for example, distal to and/or adjacent to a distal end of track 116. For example, the one or more indications 124 may include an arrow and/or textual indicators (e.g., “LOCK”). The one or more indications 124 may indicate to the user a direction or movement, such that movable portion 110 is secured relative to stationary portion 112. For example, the one or more indications 124 may indicate to the user a direction or movement of movable portion 110 such that tab 114 is positioned within locking portion(s) 118 and the needle is secured in the extended configuration. Alternatively or additionally, although not shown, the one or more indications 124 may indicate to the user a direction or movement of movable portion 110 such that 110 is removed from locking portion(s) 118, for example, to unlock movable portion 110 relative to stationary portion 112, and thus retract the needle from the extended configuration. Alternative indicators may also be used (e.g., stationary portion 112 may include a second opening, and movable portion 110 may include colored portions, such as red and green portions, that align with the second opening depending on whether movable portion 110 is secured/locked relative to stationary portion 112).

Although portions 110 and 112 of handle 102 are discussed herein as being movable and stationary respectively, it is understood that the movement may be reversed. For example, portion 110 may instead be stationary and portion 112 may be movable to control the extension and/or retraction of a needle from a distal end of shaft 104.

In use, a user may advance shaft 104 to a treatment site (e.g., through a working channel of an introducing device) with medical device 100 in the configuration of FIG. 1A (i.e., with the needle in a retracted configuration). The user may contact knob portion 120 (e.g., with a thumb or other fingers) to move movable portion 110 distally relative to stationary portion 112, thereby advancing the needle. The movement of movable portion 110 distally may cause tab 114 to ride along track 116 until it engages with locking portion 118 (the configuration of FIG. 1B). The needle may be retained/locked in the extended position, due to an interaction between tab 114 and locking portion 118. After performing a procedure with the needle, the user may then engage knob portion 120 in order to retract the needle. For example, the user may rotate knob portion 120 so that tab 114 disengages with locking portion 118. Alternatively, the user may move knob portion 120 slightly distally in order to disengage tab 114 from locking portion 118. A spring or other biasing element may cause movable portion 110 to move proximally, retracting the needle. Alternatively, the user may pull proximally on knob portion 120 to retract the needle.

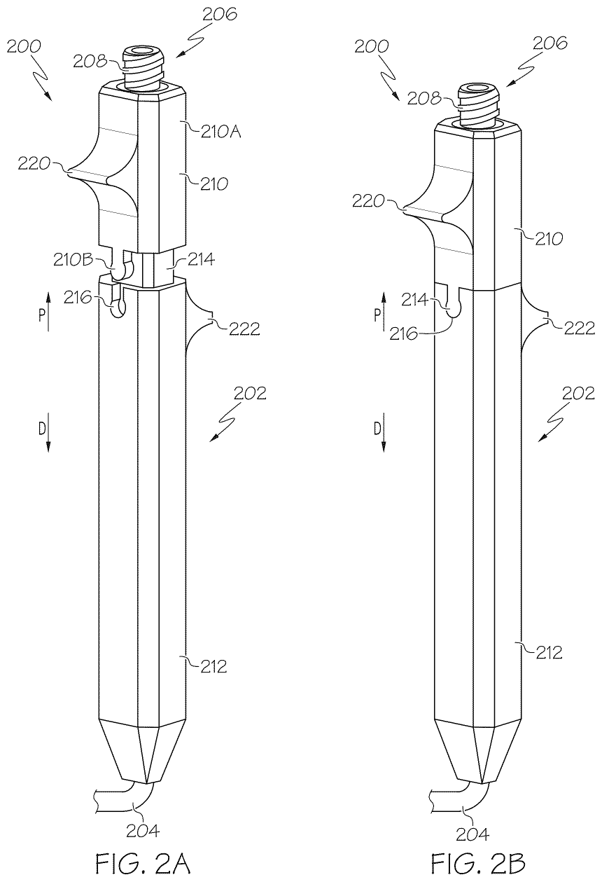

FIGS. 2A and 2B illustrate perspective views of an exemplary medical device 200 that includes a handle 202 and a shaft 204. Although not shown, shaft 204 includes a distal end, and medical device 200 further includes a movable needle or other movable element. As discussed in detail below, one or more portions of handle 202 are movable to control the movement and/or position of the needle, for example, movable between a retracted position (FIG. 2A) and an extended position (FIG. 2B). Although not shown, one or more portions of handle 202, shaft 204, and/or the needle may include one or more lumens or channels, for example, to deliver one or more fluids from one or more fluid sources (e.g., coupled to a proximal end of handle 202), through medical device 200, and out of the needle. For example, a proximal portion of handle 202 may include a coupling portion 206. Coupling portion 206 may be a port, a luer, etc., for example, including one or more threads 208, to couple a syringe or other fluid source to medical device 200.

As shown in FIGS. 2A and 2B, handle 202 includes an internal or movable portion 210 and an external or stationary portion 212. Movement of movable portion 210 relative to stationary portion 212 may control the position of the needle relative to the distal end of shaft 204. In some aspects, movable portion 210 may include a wider proximal portion 210A and a narrower distal portion 210B. For example, distal portion 210B may be extendable within a lumen or interior of stationary portion 212.

Movable portion 210 may include a projection or tab 214, and stationary portion 212 may include a channel 216 into which tab 214 may be positioned, for example, to form a snap-fit. The snap-fit interaction of tab 214 with channel 216 may help to allow the user to lock or unlock the needle as needed during the procedure (e.g., lock the needle in an extended position/unlock the needle from the extended position). The snap-fit interaction of tab 214 with channel 216 may require a small distal force to close or otherwise secure movable portion 210 relative to stationary portion 212, for example, to secure the needle in the extended configuration. Moreover, the snap-fit interaction of tab 214 with channel 216 may create audible and/or haptic feedback with secured or unsecured. Furthermore, in some aspects, the snap-fit interaction of tab 214 with channel 216 may provide an intuitive and/or easy to use feeling for the user operating handle 202. Similarly, a small proximal force may be required to remove tab 214 from channel 216, thereby freeing/opening movable portion 210 from stationary portion 212.

In some aspects, tab 214 may extend from a distal portion of wider proximal portion 210A of movable portion 210. Tab 214 may include a circular distal portion, and a straight or narrower proximal portion. Channel 216 may extend distally from a proximal end of stationary portion 212. Channel 216 may include a corresponding size and/or shape to tab 214. In some aspects, a shape of distal portion 210B of movable portion 210 may correspond to a shape of the lumen or interior of stationary portion 212. Such features may facilitate a snap-fit of tab 214 within channel 216. The corresponding shapes may help to ensure that tab 214 is aligned with channel 216.

In these aspects, positioning tab 214 in channel 216 may help to secure the position of movable portion 210 relative to stationary portion 212. Such a position of tab 214 in channel 216 correspond to an extended configuration of the needle, and thus tab 214 and channel 216 may cooperate to secure (e.g., lock) the needle in the extended configuration (FIG. 2B). Additionally, the user may urge movable portion 210 proximally relative to stationary portion 212, for example, to move tab 214 out of channel 216, in order to transition from an extended configuration of the needle (FIG. 2B) to the retracted configuration (FIG. 2A).

Furthermore, although not shown, movement of movable portion 210 relative to stationary portion 212 may engage one or more springs, for example, biasing the distal movement of movable portion 210 (e.g., bias movable portion 210 in the proximal direction). For example, as movable portion 210 moves distally relative to stationary portion 212, the one or more springs may be compressed. With tab 214 in channel 216, the position of movable portion 210 may be at least partially secured relative to stationary portion 212. Then, when movable portion 210 is manipulated (e.g., urged proximally) such that tab 214 is moved out of channel 216, the one or more springs may bias movable portion 210 to move from the extended configuration (FIG. 2B) to the retracted configuration (FIG. 2A).

In some aspects, movable portion 210 may include one or more grip portions 220, for example, on a proximal portion of movable portion 210 (e.g., adjacent to coupling portion 206, on wider proximal portion 210A). Grip portion(s) 220 may help the user grip and/or otherwise manipulate (e.g., extend/retract, rotate, etc.) movable portion 210. Furthermore, in some aspects, stationary portion 212 may include one or more grip portions 222, for example, on a proximal portion of stationary portion 212. Grip portion(s) 222 may help the user grip and/or hold stationary portion 212. Additionally, grip portion(s) 222 on stationary portion 212 may be positioned on an opposite side from grip portion(s) 220 on movable portion 210, which may help to provide for movement of movable portion 210 relative to stationary portion 212 with a single hand of the user. By gripping a proximal portion of grip portion 220 and a distal portion of grip portion 222 and moving the fingers together (e.g., applying a force to at least one of grip portion 220 or grip portion 222), a user may move movable portion 210 distally relative to stationary portion 212 in order to close/lock handle 202 (and advance/secure the needle distally). By gripping a distal portion of grip portion 220 and a proximal portion of grip portion 222 and moving the fingers apart, a user may move movable portion 210 proximally relative to stationary portion 212 in order to open/unlock handle 202 (and move the needle proximally).

In use, a user may advance shaft 204 to a treatment site (e.g., through a working channel of an introducing device) with medical device 200 in the configuration of FIG. 2A (i.e., with the needle in a retracted configuration). The user may engage grip portions 220 and/or 222 to move movable portion 210 distally relative to stationary portion 212, thereby advancing the needle. The movement of movable portion 110 distally may cause tab 214 to engage with channel 216. The needle may be retained/locked in the extended position, due to an interaction between tab 214 and channel 216, as shown in FIG. 2B. After performing a procedure with the needle, the user may then engage knob grip portions 220 and/or 222 in order to move movable portion 210 proximally and retract the needle. A spring or other biasing element may cause movable portion 210 to move proximally, retracting the needle. Alternatively, the user may pull proximally on grip portions 220 to move movable portion 210 proximally and to retract the needle.

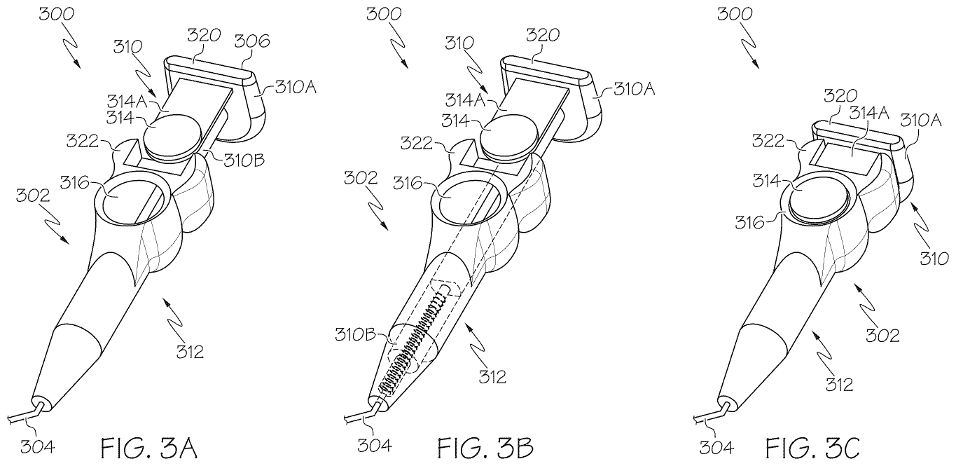

FIGS. 3A-3C illustrate perspective views of an exemplary medical device 300 that includes a handle 302 and a shaft 304. Although not shown, shaft 304 includes a distal end, and medical device 300 further includes a movable needle. As discussed in detail below, one or more portions of handle 302 are movable to control the movement and/or position of the needle, for example, movable between a retracted position (FIGS. 3A and 3B) and an extended position (FIG. 3C). Although not shown, one or more portions of handle 302, shaft 304, and/or the needle may include one or more lumens or channels, for example, to deliver one or more fluids from one or more fluid sources (e.g., coupled to a proximal end of handle 302), through medical device 300, and out of the needle. For example, a proximal portion of handle 302 may include a coupling portion 306 (FIG. 3A). Coupling portion 306 may be a port, a luer, etc., for example, including one or more threads (not shown), to couple a syringe or other fluid source to medical device 300.

As shown in FIGS. 3A-3C, handle 302 includes an internal or movable portion 310 and an external or stationary portion 312. Stationary portion 312 is shown as partially transparent or see-through in FIG. 3B. Movement of movable portion 310 relative to stationary portion 312 may control the position of the needle relative to the distal end of shaft 304. In some aspects, movable portion 310 may include a wider proximal portion 310A and a narrower distal portion 310B. For example, distal portion 310B may be extendable within a lumen or interior of stationary portion 312, as shown in FIG. 3B. Movable portion 310 also includes a push button or tab 314. Tab 314 may be formed by or otherwise coupled to a cantilevered projection, for example, coupled to proximal portion 310A of movable portion 310 by an extension portion 314A, for example, extending distally from proximal portion 310A of movable portion 310 to tab 314. Extension portion 314A may extend approximately parallel to narrower distal portion 310B. Tab 314 may be a circular tab extending radially from extension portion 314A. Stationary portion 312 may include an opening 316, for example, into which tab 314 may be positioned, for example, to form a snap-fit. For example, in some aspects, tab 314 may be generally cylindrical (e.g., extending laterally away from extension portion 314A), and opening 316 may be generally circular (e.g., sized and/or shaped to receive a portion of tab 314).

The snap-fit interaction of tab 314 with opening 316 may help to allow the user to lock or unlock the extension of needle as needed during the procedure. The snap-fit interaction of tab 314 with opening 316 may require a small force to close or otherwise secure movable portion 310 relative to stationary portion 312, for example, to secure the needle in the extended configuration. Moreover, the snap-fit interaction of tab 314 with opening 316 may create audible and/or haptic feedback with secured or unsecured. Furthermore, in some aspects, the snap-fit interaction of tab 314 with opening 316 may provide an intuitive and/or easy to use feeling for the user operating handle 302.

In these aspects, positioning tab 314 in opening 316 may help to secure the position of movable portion 310 relative to stationary portion 312. When tab 314 is positioned in opening 316, the needle may be in an extended configuration. Thus, positioning tab 314 in opening 316 may help to secure the needle in the extended configuration (FIG. 3C). Additionally, the user may depress or otherwise urge tab 314 downward (e.g., toward the longitudinal axis of handle 302/narrower distal portion 310B), for example, to move or otherwise transition tab 314 out of opening 316, in order to transition from an extended configuration of the needle (FIG. 3C) to the retracted configuration (FIGS. 3A and 3B).

Furthermore, as shown in FIG. 3B, movement of movable portion 310 relative to stationary portion 312 may engage one or more springs 330, for example, biasing the distal movement of movable portion 310 (e.g., bias movable portion 310 in the proximal direction). For example, as movable portion 310 moves distally relative to stationary portion 312, the one or more springs 330 may be compressed. With tab 314 in opening 316, the position of movable portion 310 may be at least partially secured relative to stationary portion 312 by an engagement between tab 314 and opening 316. Then, when movable portion 310 is manipulated such that tab 314 is moved out of opening 316 (e.g., depressed), the one or more springs 330 may bias movable portion 310 to move from the extended configuration (FIG. 3C) to the retracted configuration (FIGS. 3A and 3B).

In some aspects, movable portion 310 may include one or more grip portions 320, for example, on a proximal portion of movable portion 310 (e.g., adjacent to coupling portion 306). Grip portion(s) 320 may help the user grip and/or otherwise manipulate (e.g., extend/retract, rotate, etc.) movable portion 310. Furthermore, in some aspects, stationary portion 312 may include one or more grip portions 322, for example, on a proximal portion of stationary portion 312. Grip portion(s) 322 may help the user grip and/or hold stationary portion 312, which may help to provide for movement of movable portion 310 relative to stationary portion 312 with a single hand of the user.

In use, a user may advance shaft 304 to a treatment site (e.g., through a working channel of an introducing device) with medical device 300 in the configuration of FIGS. 3A and 3B (i.e., with the needle in a retracted configuration). The user may engage grip portions 320 and/or 322 to move movable portion 310 distally relative to stationary portion 312, thereby advancing the needle. The movement of movable portion 310 distally may cause tab 314 to move distally until it engages with opening 316. For example, extension portion 314A may be resilient/have shape memory properties, such that once tab 314 is positioned within opening 316, tab 314 moves outward (away from a longitudinal axis of handle 302), through opening 316. The needle may be retained/locked in the extended position, due to an interaction between tab 314 and opening 316, as shown in FIG. 3C. After performing a procedure with the needle, the user may then press inward/down on tab 314 to disengage tab 314 from opening 316, in order to move movable portion 310 proximally and retract the needle. A spring or other biasing element may cause movable portion 310 to move proximally, retracting the needle. Alternatively, the user may pull proximally on grip portions 320 to move movable portion 310 proximally and to retract the needle.

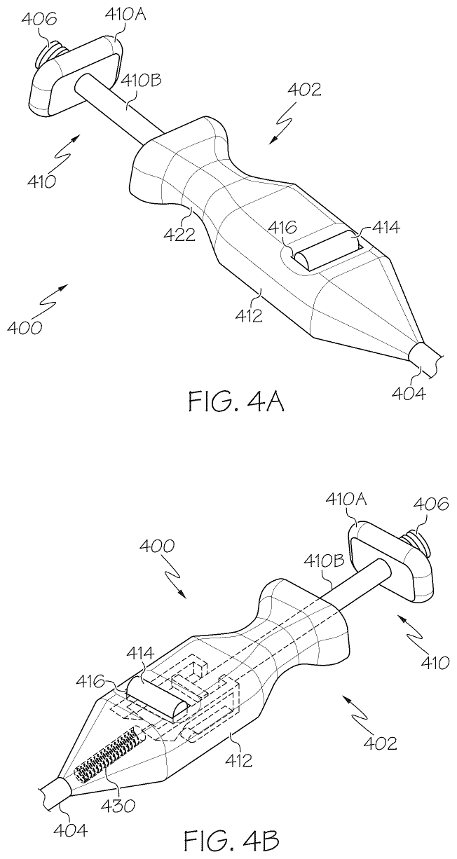

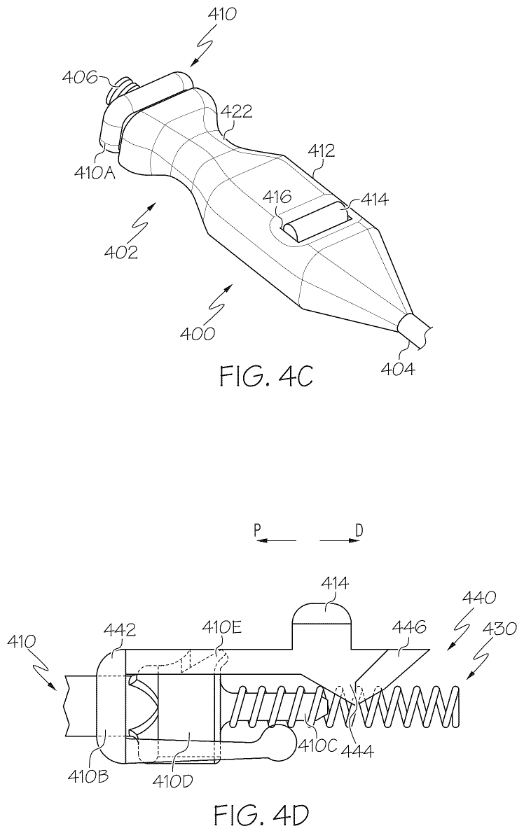

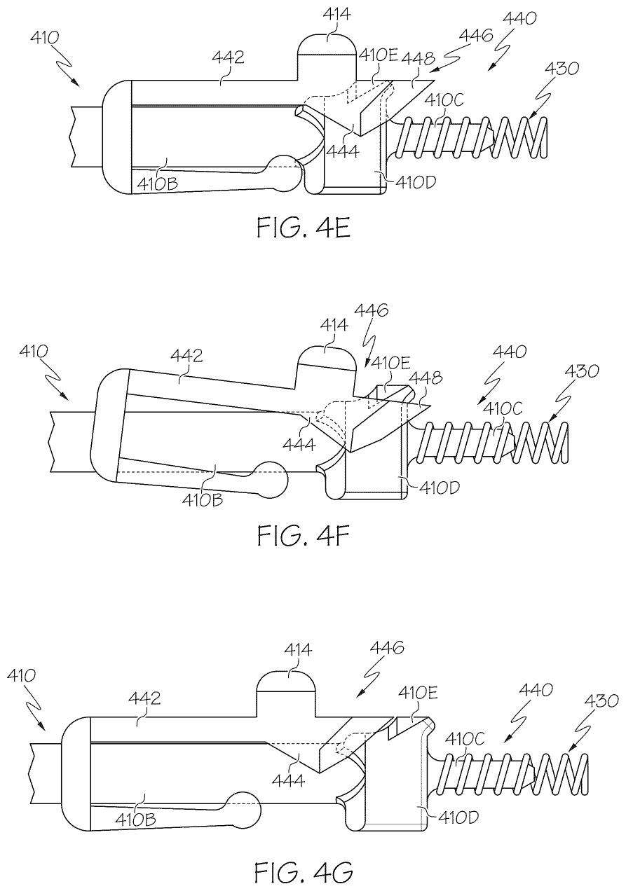

FIGS. 4A-4C illustrate perspective views of an exemplary medical device 400 that includes a handle 402 and a shaft 404. FIGS. 4D-4F illustrate internal mechanisms of handle 402 in various configurations. Although not shown, shaft 404 includes a distal end, and medical device 400 further includes a movable needle or other movable element. As discussed in detail below, one or more portions of handle 402 are movable to control the movement and/or position of the needle, for example, movable between a retracted position (FIGS. 4A and 4B) and an extended position (FIG. 4C). Although not shown, one or more portions of handle 402, shaft 404, and/or the needle may include one or more lumens or channels, for example, to deliver one or more fluids from one or more fluid sources (e.g., coupled to a proximal end of handle 402), through medical device 400, and out of the needle. For example, a proximal portion of handle 402 may include a coupling portion 406. Coupling portion 406 may be a port, a luer, etc., for example, including one or more threads, to couple a syringe or other fluid source to medical device 400.

As shown in FIGS. 4A-4C, handle 402 may be like a syringe handle, and may include an internal or movable portion 410 and an external or stationary portion 412. Stationary portion 412 is shown as partially transparent or see-through in FIG. 4B. Movement of movable portion 410 relative to stationary portion 412 may control the position of the needle relative to the distal end of shaft 404. In some aspects, movable portion 410 may include a wider proximal portion 410A and a narrower distal portion 410B. For example, distal portion 410B may be extendable within a lumen or interior of stationary portion 412, as shown in FIG. 4B. Movable portion 410 also includes or otherwise interacts with a push button or tab 414. Tab 414 may be formed by a cantilevered projection, for example, movable relative to stationary portion 412 based on the position of movable portion 410. Stationary portion 412 may include an opening 416, for example, into which a portion of tab 414 may be positioned, for example, to form a snap-fit. For example, in some aspects, tab 414 may be generally rectangular, and opening 416 may be generally rectangular (e.g., sized and/or shaped to receive a portion of tab 414). As discussed below, tab 414 may be movable (e.g., pivotable) based on movement (e.g., longitudinal movement) of movable portion 410, but tab 414 may not be connected to or otherwise be a part of movable portion 410.

The snap-fit interaction of tab 414 with opening 416 may help to allow the user to lock or unlock the extension of needle as needed during the procedure. The snap-fit interaction of tab 414 with opening 416 may require a small force to close or otherwise secure movable portion 410 relative to stationary portion 412, for example, to secure the needle in the extended configuration. Moreover, the snap-fit interaction of tab 414 with opening 416 may create audible and/or haptic feedback with secured or unsecured. Furthermore, in some aspects, the snap-fit interaction of tab 414 with opening 416 may provide an intuitive and/or easy to use feeling for the user operating handle 402.

In these aspects, tab 414 may be movable within opening 416, for example, to help to secure the position of movable portion 410 relative to stationary portion 412. Furthermore, tab 414 within opening 416 may help to secure the needle in the extended configuration (FIG. 4C). Additionally, as discussed below with respect to FIGS. 4D-4G the user may depress or otherwise urge tab 414 downward (e.g., toward the longitudinal axis of handle 402), for example, in order to transition from an extended configuration of the needle (FIG. 4C) to the retracted configuration (FIGS. 4A and 4B). Moreover, tab 414 may be connected to one or more biasing elements, for example, to be biased toward the upward (e.g., away from the longitudinal axis of handle 402).

Furthermore, as shown in FIGS. 4B and 4D-4G, movement of movable portion 410 relative to stationary portion 412 may engage one or more springs 430, for example, biasing the distal movement of movable portion 410 (e.g., bias movable portion 410 in the proximal direction). Spring 430 may extend generally in a proximal/distal direction. As movable portion 410 moves distally relative to stationary portion 412, the one or more springs 430 may be compressed. With tab 414 in opening 416, the position of movable portion 410 may be at least partially secured relative to stationary portion 412. Then, when movable portion 410 is manipulated such that tab 414 is moved out of opening 416 (e.g., depressed), the one or more springs 430 may bias movable portion 410 to move from the extended configuration (FIG. 4C) to the retracted configuration (FIGS. 4A and 4B) (i.e., may bias movable portion 410 to move proximally).

In some aspects, stationary portion 412 may include one or more grip portions 422, for example, on a proximal portion of stationary portion 412. Grip portion(s) 422 may help the user grip and/or hold stationary portion 412, which may help to provide for movement of movable portion 410 relative to stationary portion 412 with a single hand of the user.

FIGS. 4D-4G illustrate internal portions of handle 402 (FIGS. 4A-4C) in various configurations. Specifically, FIGS. 4D-4G illustrate a trigger mechanism 440, including movable portion 410, tab 414, and spring 430. As discussed above, distal portion 410B of movable portion 410 may extend into handle 402. Additionally, movable portion 410 may include a narrow portion 410C and a widened portion 410D. Narrow portion 410C may form a distalmost portion of movable portion 410, for example, distal to widened portion 410D. Narrow portion 410C may at least partially overlap with one or more springs 430, for example, extending within a central lumen of spring 430 and movable relative to spring 430, as shown in FIGS. 4D-4G. Moreover, as discussed below, widened portion 410D may interact with tab 414. For example, tab 414 may extend from a trigger body 442 such that movement of one or more of movable portion 410, tab 414, and/or spring 430 may lock or otherwise secure movable portion 410, and thus a position of a needle, in one or more an extended configuration or a retracted configuration.

FIG. 4D illustrates an unlocked or retracted configuration, for example, such that the needle at the distal end of shaft 404 (FIGS. 4A-4C) is in a retracted configuration. For example, movable portion 410 and widened portion 410D are retracted relative to trigger mechanism 440, and spring 430 is in an expanded configuration. In these aspects, widened portion 410D of movable portion 410 is biased proximally by spring 430. Additionally, trigger mechanism 440, including tab 414, may bias tab 414 and other internal components toward the configuration shown in FIG. 4D.

FIG. 4E illustrates movable portion 410 being extended or otherwise urged distally, for example, to extend the needle. As shown, widened portion 410D of movable portion 410 includes a sloped portion or an extension 410E, and trigger body 442 also includes a sloped portion or prong 444. Prong 444 may be positioned internally to handle 402, for example, on an opposite side of a trigger extension 446 from tab 414. As movable portion 410 is extended or urged distally, widened portion 410D (e.g., extension 410E) may abut or otherwise interact with prong 444, for example, with a sloped projection 448. For example, a distal face or portion of extension 410E may abut or otherwise engage with a proximal face or portion of sloped projection 448. In some aspects, widened portion 410D may push prong 444 such that trigger extension 446 is bent or otherwise moved (e.g., downward in FIG. 4E).

For example, FIG. 4F illustrates movable portion 410 being extended or otherwise urged distally relative to the configuration shown in FIG. 4E. As shown, widened portion 410D (e.g., extension 410E) may engage with sloped projection 448 and urge trigger extension 446 downward. With trigger extension 446 bent or moved, widened portion 410D and the other portions of movable portion 410 may be extended distally, for example, to extend the needle. Extending movable portion 410 distally may also compress spring 430. Moreover, with widened portion 410D interacting with prong 444 and trigger extension 446, tab 414 may also be urged or moved (e.g., downward in FIG. 4F).

FIG. 4G illustrates movable portion 410 secured in the extended position, for example, with the needle in an extended configuration. As mentioned above, trigger mechanism 440, including tab 414, may bias tab 414 and other internal components toward the configuration shown in FIG. 4D. Accordingly, after widened portion (e.g., extension 410E) is distal to sloped projection 448, trigger mechanism 440 returns to the original configuration. As shown, a proximal portion of widened portion 410D (e.g., a proximal face of portion of extension 410E) may abut or otherwise interact with prong 444 of trigger body 442 (e.g., with a distal face or portion of sloped projection 448). For example, prong 444 and sloped projection 448 may help to prevent widened portion 410D, and thus movable portion 410, from moving proximally (e.g., due to a force from spring 430) to return movable portion 410 and the needle to the retracted configuration. Moreover, the user may manipulate tab 414 to control the position or movement of trigger body 442 and prong 444. For example, the user manipulate tab 414 to move prong 444 and sloped projection 448 downward (e.g., pushing downward and/or urging distally in FIG. 4G) such that prong 444 and sloped projection 448 no longer impedes or restricts the proximal movement of movable portion 410. In these aspects, spring 430 may bias widened portion 410D proximally, for example, to return movable portion 410 and the needle from the extended configuration to the retracted configuration. These steps may be repeated as many times as necessary during the medical procedure.

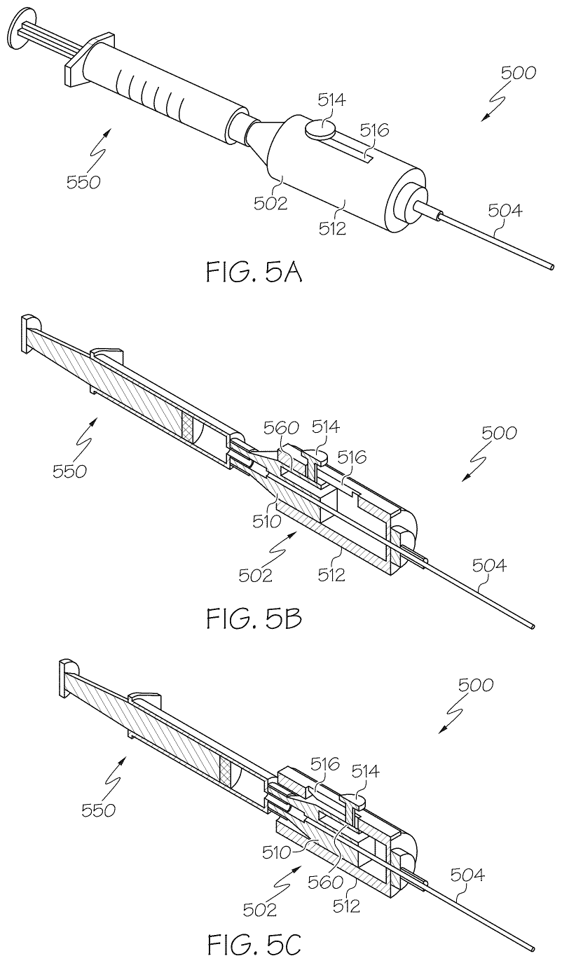

FIGS. 5A-5C illustrate various view of an exemplary medical device 500 that includes a handle 502 and a shaft 504. FIG. 5A is a perspective view of medical device 500, including a syringe 550 coupled to a proximal end of handle 502, with the medical device in a retracted configuration. FIGS. 5B and 5C are cross-sectional views of medical device 500 in the retracted configuration and an extended configuration, respectively. Although not shown, shaft 504 includes a distal end, and medical device 500 further includes a movable needle or other movable element. As discussed in detail below, one or more portions of handle 502 are movable to control the movement and/or position of the needle, for example, movable between a retracted position (FIGS. 5A and 5B) and an extended position (FIG. 5C). Moreover, as shown, one or more portions of handle 502, shaft 504, and/or the needle may include one or more lumens or channels, for example, to deliver one or more fluids from one or more fluid sources (e.g., syringe 550), through medical device 100, and out of the needle, as discussed above.

Handle 502 includes an internal or movable portion 510 (See. FIGS. 5B and 5C) and an external or stationary portion 512, for example, in a slider handle arrangement. Movement of movable portion 510 relative to stationary portion 512 may control the position of the needle relative to the distal end of shaft 504. For example, movable portion 510 may include a projection, button, or tab 514, and stationary portion 512 may include a channel or track 516, for example, through which tab 514 may move as movable portion 510 moves relative to stationary portion 512. Track 516 maybe formed by an opening (e.g., carved or otherwise extending radially through) in one or more portions of stationary portion 512 of handle 502. The interaction of tab 514 within track 516 may limit the longitudinal movement of movable portion 510 relative to stationary portion 512 of handle. For example, with tab 514 in a proximal sections of track 516 (e.g., in FIGS. 5A and 5B), the needle may be in the retracted configuration. Additionally, with tab 514 in a distal section of track 516 (e.g., in FIG. 5C), the needle may be in the extended configuration. Moreover, track 516 may include one or more slots or grooves (e.g., wider portions) at the proximal and/or distal sections. Tab 514 may be positioned within the slots or grooves to help secure tab 514 (and thus movable portion 510 and the needle) in the position and corresponding configurations. The extension and retraction may be repeated as many times as necessary during the medical procedure.

In some aspects, tab 514 may only be movable within track 516 when depressed (e.g., radially inward). For example, tab 514 may be formed of a deformable or bendable material (e.g., plastic, metal, etc.). Tab 514 may extend from movable portion 510 by a cantilevered extension 560 (see, e.g., FIGS. 5B and 5C). When tab 514 is not depressed, tab 514 and/or cantilevered extension 560 may interact with an internal surface of stationary portion 512, such that a frictional engagement may help to secure the position of movable portion 510 relative to stationary portion 512. For example, a portion of the internal surface of stationary portion 512 adjacent to one or more portions of track 516 may frictionally engage with tab 514 and/or cantilevered extension 560. Depression of tab 514 may at least partially deform the material of tab 514 and/or cantilevered extension 560, reducing the frictional engagement, and allowing for relative movement of movable portion 510 relative to stationary portion 512. Although not shown, in some aspects, handle 502 may include one or more springs or biasing elements, for example, positioned adjacent to and/or radially within (i.e., below in FIG. 5B) tab 514. In these aspects, the one or more springs may help to augment and/or adjust a deformability of the material that forms tab 514. For example, the one or more springs may help to bias tab 514 radially outward, for example, to help cantilevered extension 560 engage with the internal surface of stationary portion 512.

Additionally, although not shown, medical device 500 may include one or more springs. The one or more springs may be positioned distal to movable portion 510 and may bias movable portion 510 proximally, for example, toward the retracted configuration from the extended configuration. Additionally, although not shown, stationary portion 512 may include one or more indications, for example, adjacent to one or more portions of track 516, indicating an extended or retracted configuration of the needle.

In use, a user may advance shaft 504 to a treatment site (e.g., through a working channel of an introducing device) with medical device 500 in the configuration of FIGS. 5A and 5B (i.e., with the needle in a retracted configuration). The user may engage tab 514 (e.g., depress and advance tab 514 within track 516) to move movable portion 510 distally relative to stationary portion 512, thereby advancing the needle. Tab 514 may be secured in one or more portions of track 516, for example, via one or more slots or grooves in track 516 and/or via the frictional engagement of tab 514 and/or cantilevered extension 560 with an internal surface of stationary portion 512. After performing a procedure with the needle, the user may then engage tab 514 (e.g., depress and retract tab 514 within track 516) to move movable portion 510 proximally relative to stationary portion 512, and thereby retracting the needle. A spring or other biasing element may cause movable portion 510 to move proximally, retracting the needle. Alternatively, the user may manipulate tab 514 and/or pull movable portion 510 proximally to move movable portion 510 proximally and to retract the needle.

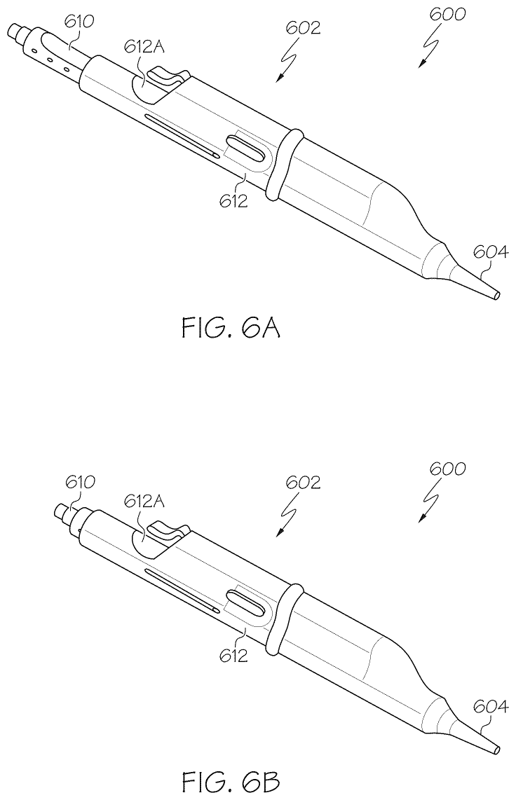

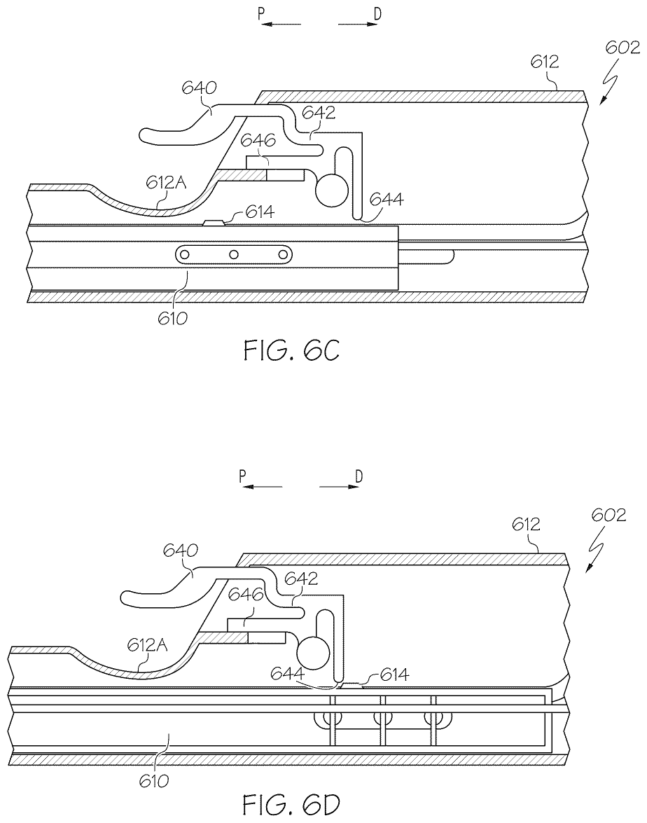

FIGS. 6A-6D are various views of another exemplary medical device 600 that includes a handle 602 and a shaft 604. FIGS. 6A and 6B illustrate handle 602 in a retracted configuration and an extended configuration, respectively. FIGS. 6C and 6D illustrate internal components of handle 602 in the retracted configuration and the extended configuration, respectively. One or more portions of handle 602, shaft 604, and/or the needle may include one or more lumens or channels, for example, to deliver one or more fluids from one or more fluid sources (e.g., coupled to a proximal end of handle 602), through medical device 600, and out of the needle. For example, a proximal portion of handle 602 may include a coupling portion, which may be a port, a luer, etc., to couple a syringe or other fluid source to medical device 600.

As shown in FIGS. 6A-6D, handle 602 includes a movable portion 610 and a stationary portion 612, and as discussed above, movement of movable portion 610 relative to stationary portion 612 may control the position of the needle or other movable element (e.g., between at least a retracted position and an extended position). Moreover, movable portion 610 may include a tab 614 (FIGS. 6C and 6D), and stationary portion 612 may include a trigger 640 that may interact with tab 614 to help to secure the position of movable portion 610 relative to stationary portion 612, for example, in the extended configuration. In these aspects, trigger 640 may a part of or otherwise coupled to a trigger body 642, and trigger body 642 may include an extension or prong 644 that interacts with tab 614. For example, with movable portion 610 in a retracted configuration, tab 614 may be proximal of prong 644, as shown FIG. 6C. Then, when movable portion 610 is extended distally to transition the needle to the extended configuration, tab 614 may pass under and/or otherwise be positioned distally past prong 644, as shown in FIG. 6D. For example, a distal portion of tab 614 may include an angled or ramped face, which may interact with prong 644 to push prong 644 upward to allow for tab 614 and movable portion 610 to pass distally of prong 644. Additionally, a proximal portion of tab 614 may include a flat (e.g., perpendicular to a longitudinal axis of movable portion 610) face, which may interact with prong 644 such that the proximal portion of tab 614 abuts prong 644 and inhibit tab 614 and movable portion 610 from moving back proximally of prong 644. Accordingly, prong 644 may abut or otherwise interact with tab 614 to help retain movable portion 610 (and thus the needle) in the extended configuration.

In some aspects, stationary portion 612 may include a depression 612A, for example, to accommodate or otherwise allow for the movement of trigger 640. Furthermore, trigger 640 may be pivotably coupled to stationary portion 612, for example, biased toward the configuration shown in FIGS. 4C and 4D by a trigger spring 646 or other biasing mechanism. For example, various aspects of trigger 640 and/or trigger body 642 may pivot about a pivot point when trigger 640 is depressed. Additionally, when trigger 640 is depressed (e.g., radially inward), prong 644 may move (e.g., upward in FIGS. 6C and 6D), which may allow for tab 614 and movable portion 612 to move proximally, for example, to transition from the extended configuration to the retracted configuration. In other words, when trigger 640 is not depressed, prong 644 may block tab 614 from moving proximally. When trigger 640 is depressed, prong 644 may be raised such that it no longer blocks tab 614. The transitions to and/or from the retracted and extended configurations may be repeated as many times as necessary during the medical procedure.

Although not shown, handle 602 may include one or more springs or other biasing elements, as discussed above, such that distal movement of movable portion 610 is biased proximally. Accordingly, when trigger 640 is depressed and prong 644 moves, the one or more springs may bias movable portion 612 proximally such that movable portion 612 and tab 614 may move proximally past prong 644 and trigger 640 to transition from the extended configuration to the retracted configuration.

In use, a user may advance shaft 604 to a treatment site (e.g., through a working channel of an introducing device) with medical device 500 in the configuration of FIGS. 6A and 6C (i.e., with the needle in a retracted configuration). The user may urge movable portion 610 distally relative to stationary portion 612, thereby advancing the needle. Tab 614 may be positioned distal to prong 644, which may secure the position of movable portion 610 relative to stationary portion 612 (e.g., with the needle extended). After performing a procedure with the needle, the user may then depress trigger 640, which may raise prong 644. Raising prong 644 thus allows tab 614 and movable portion 610 to retract proximally relative to stationary portion 612 and retracting the needle. A spring or other biasing element may cause movable portion 610 to move proximally, retracting the needle. Alternatively, the user may pull movable portion 610 proximally to move movable portion 610 proximally and to retract the needle.

While principles of this disclosure are described herein with reference to illustrative aspects for various applications, it should be understood that the disclosure is not limited thereto. Those having ordinary skill in the art and access to the teachings provided herein will recognize additional modifications, applications, aspects, and substitution of equivalents all fall within the scope of the aspects described herein. Accordingly, the disclosure is not to be considered as limited by the foregoing description.

We claim:

1. A medical device, comprising:
a handle, including:
a movable portion; and
a stationary portion;
a shaft extending from a distal portion of the handle, wherein a distal portion of the shaft includes a needle;
wherein movement of the movable portion of the handle relative to the stationary portion of the handle controls an extension or retraction of the needle, and
wherein the movable portion includes a tab, wherein a position of the tab relative to the stationary portion releasably secures a position of the movable portion relative to the stationary portion and also secures a position of the needle relative a distal end of the shaft.

2. The medical device of claim 1, wherein the stationary portion includes a track to movably receive a portion of the tab.

3. The medical device of claim 2, wherein the track includes one or more locking portions.

4. The medical device of claim 1, further comprising a trigger, wherein the trigger is pivotable relative to the stationary portion of the handle.

5. The medical device of claim 4, wherein the trigger is configured to interact with the tab of the movable portion to limit proximal movement of the movable portion.

6. The medical device of claim 1, further comprising one or more springs, wherein the one or more springs bias the distal movement of the movable portion relative to the stationary portion of the handle.

7. The medical device of claim 1, wherein the movable portion further comprises a first grip portion configured to accept an applied force, the applied force causing the movable portion to move relative to stationary portion.

8. The medical device of claim 7, wherein the movable portion is disposed on a first face, wherein the stationary portion further comprises a second grip portion disposed on a second face, the second face opposite the first face.

9. The medical device of claim 3, wherein the tab comprises is circular and extends radially from an extension portion.

10. The medical device of claim 9, wherein the one or more locking portions comprise a circular opening configured to engage the tab.

11. The medical device of claim 10, wherein force applied to the tab causes the tab to release from the recessed opening.

12. The medical device of claim 1, wherein the tab comprises a cantilevered projection disposed within an opening on the stationary portion.

13. The medical device of claim 12, wherein a position of the tab and the opening define a range of movement of the needle.

14. The medical device of claim 1 wherein the movable portion is configured to mate with the stationary portion.

15. The medical device of claim 6, wherein the one or more springs are biased by one or more of the movable portion, the stationary portion, or a trigger, wherein the trigger is pivotable relative to the stationary portion of the handle.

16. A medical device, comprising:
a handle, including:
a movable portion; and
a stationary portion;
a shaft extending from a distal portion of the handle, wherein a distal portion of the shaft includes a needle;
wherein movement of the movable portion of the handle relative to the stationary portion of the handle controls a position of the needle, and
wherein the movable portion includes a tab, wherein a position of the tab relative to the stationary portion releasably locks a position of the movable portion relative to the stationary portion.

17. The medical device of claim 16, wherein the tab further locks a position of the needle relative a distal end of the shaft.

18. The medical device of claim 16, further comprising a trigger, wherein the trigger is pivotable relative to the stationary portion of the handle, wherein the handle includes a depression configured to receive the trigger, wherein the trigger comprises a prong configured to engage the tab.

19. A medical device, comprising:
a handle, including:
a movable portion comprising a knob; and
a stationary portion;
a shaft extending from a distal portion of the handle, wherein a distal portion of the shaft includes a needle;
wherein movement of the movable portion of the handle relative to the stationary portion of the handle controls an extension or retraction of the needle, and
wherein the movable portion includes a tab, wherein a position of the tab relative to the stationary portion releasably secures a position of the movable portion relative to the stationary portion and also secures a position of the needle relative a distal end of the shaft, wherein rotation of the knob rotates the movable portion relative to the stationary portion.

20. The medical device of claim 19, wherein the stationary portion includes a track to movably receive a portion of the tab, wherein the track is angled relative to a longitudinal axis of the stationary portion.
